(19) United States
(12) Patent Application Publication
(10) Pub. No.: US 2023/0212505 A1
(43) Pub. Date: Jul. 6, 2023

(54) Dried Fish Fermentation Process and Starter Culture Development Technology

(71) Applicants: Zhejiang Gongshang University, Hangzhou (CN); Shaoxing baima Lake Food Co. LTD, Shaoxing City (CN)

(72) Inventors: Renyao JIN, Hangzhou (CN); Jiacheng YANG, Hangzhou (CN); Lihua CHEN, Shaoxing City (CN); Yanling SONG, Hangzhou (CN); Xiaoxia LIU, Hangzhou (CN)

(21) Appl. No.: 17/654,393

(22) Filed: Mar. 10, 2022

(30) Foreign Application Priority Data
Dec. 31, 2021 (CN) 2021116827828

(51) Int. Cl. C12N 1/20; A23L 17/00

(52) U.S. Cl. CPC C12N 1/20; A23L 17/65; A23Y 2300/49; A23Y 2220/03; A23Y 2220/17; A23Y 2240/75; A23V 2002/00

(57) ABSTRACT

The present invention provides a dried fish fermentation process and a starter culture development technology. A mixed starter culture including Bifidobacterium species, Lactobacillus acidophilus, Lactobacillus casei, Streptococcus thermophilus and Bifidobacterium lactis is prepared for the mixed fermentation of a dried fish; and salt content in the dried fish is controlled to control a proportional relation of flora in the mixed starter culture and to optimize the fermentation process, thereby preparing more fresh and delicious, fishy-free, more soft and delicate dried fish. Therefore, the dried fish is more popular with consumers.

### TECHNICAL FIELD

The present invention belongs to the field of aquatic food processing, and particularly relates to a dried fish fermentation process and a starter culture development technology.

### BACKGROUND

Dried fish, a kind of aquatic product further processed by freshwater fish, is produced by seasoning or pickling freshwater fish in wine after being pickled by salt. Dried fish has a good appearance and good taste, chewable and thus, is deeply loved by people around Jiangsu and Zhejiang provinces. But the existing dried fish is not enough delicious and fresh, has a fishy smell and tastes hard, and thus, hard to satisfy the demands of partial consumers.

Lactic acid bacteria are generic terms of gram-positive bacteria capable of fermenting saccharides to produce lactic acid. Lactic acid bacteria usually serve as a starter culture to ferment food to give products a unique fermentation flavor. Lactic acid bacteria are widely used in fermented dairy products, vegetable products and meat products, which can not only improve food flavor, but also can inhibit putrefying bacteria and pathogenic bacteria in food, thus extending the shelf life of food. Some research reports have indicated that this is because lactic acid bacteria can produce antibacterial substances in food via a competitive effect, such as, organic acids, bacteriocins. Mixed lactobacillus fermentation is closer to original ecology by means of cooperative interaction among different flora. Lactobacillus fermentation technology has been widely applied in the fermentation of dairy products and starch-based products. But, lactic acid bacteria have been not extensively applied in low-salt fish products.

To solve the problems of high salt content, not enough delicious and fresh meat, it is urgent to find out a preparation method of dried fish with obviously improved aroma and fresh taste, preferred flavor and better taste.

### SUMMARY

To solve the above problems, the present invention provides a low-salt dried fish fermentation technology and a starter culture development technology. A mixed starter culture including Bifidobacterium species, Lactobacillus acidophilus, Lactobacillus casei, Streptococcus thermophilus and Bifidobacterium lactis is prepared for the mixed fermentation of a dried fish; and salt content in the dried fish is controlled to control a proportional relation of flora in the mixed starter culture and to optimize the fermentation process, thereby preparing more fresh and delicious, fishy-free, more soft and delicate dried fish. In particular, flavor substances, 5-hexyldihydro-2(3H)-furanone, 3-heptyldihydro-5-methyl-2(3H)-furanone, nonanal, hexadecanal as well as delicious substances, such as, glutamic acid and aspartic acid in the dried fish have obviously improved contents, such that the dried fish is more popular with consumers.

In this present invention, dried fish is fermented by a mixed starter culture; and the mixed starter culture contains lots of lactic acid bacteria. Lactic acid bacteria are utilized for the fermentation of dried fish to metabolize carbohydrates to produce a large number of lactic acids, acetic acids and substances. These metabolites can effectively reduce the fishy smell of fish products. Lactic acid bacteria can further give products a unique fermentation flavor due to fermentation. Lactic acid bacteria can make use of carbohydrates to produce organic acids; these organic acids, such as, citric acid, malic acid and acetic acid not only have fragrances, but also can be reacted with alcohol substances in fish meat to generate esters having an aromatic flavor. Lactic acid bacteria can also produce a large number of amino acids and polypeptides via the combined action between protease by itself and endogenous enzymes in fish. Moreover, lactic acid bacteria can degrade fatty acids by metabolism to produce short-chain fatty acids, thereby producing a special flavor. Fish is rich in proteins, unsaturated fatty acids and other nutrients, and thus, is susceptible to microbial contamination, leading to decomposition and corrosion. Lactic acid bacteria can effectively extend the shelf life of fish products.

Through a great number of studies, the present invention particularly discloses a formula for a mixed starter culture of dried fish. The dried fish fermented by the mixed starter culture has obviously improved flavor and delicious substances, and tastes softer and very good.

In one aspect, the present invention provides a mixed starter culture of dried fish; the mixed starter culture includes Bifidobacterium species, Lactobacillus acidophilus, Lactobacillus casei, Streptococcus thermophilus and Bifidobacterium lactis.

The research shows that compared with the fermentation of a single lactic acid bacterium, the mixed fermentation of multiple bacteria can improve the flavor and taste of the dried fish more significantly, and can further reduce fishy smell, making the dried fish more soft and delicate.

Further, in the mixed starter culture, Bifidobacterium species, Lactobacillus acidophilus, Lactobacillus casei, Streptococcus thermophilus and Bifidobacterium lactis have a volume ratio of 1:1:1:2; and the mixed starter culture has a concentration of bacterium of 10⁸-10⁹ cfu/mL.

In the five bacteria of Bifidobacterium species, Lactobacillus acidophilus, Lactobacillus casei, Streptococcus thermophilus and Bifidobacterium lactis, Bifidobacterium lactis has particularly obvious effects. Moreover, in the mixed starter culture, when the content of Bifidobacterium lactis is higher than that of other bacteria, the five bacteria exert better synergy in the mixed fermentation of dried fish. The prepared dried fish has a better flavor, higher content of flavor substances and delicious substances, and more soft taste.

In another aspect, the present invention provides a preparation method of a dried fish, mainly including the following steps:

(1) preparing a dried fish;

(2) rinsing the dried fish with fresh water; and

(3) inoculating with the mixed starter culture of claim 1 or 2 for fermentation.

Further, the step of rinsing with fresh water in the step (2) is as follows: subjecting the dried fish to desalting treatment for 1-2 h, and then baking the dried fish at a condition of 40° C., and after baking, the weight of the dried fish is 1.1-1.2 times the original weight.

Rinsing with fresh water may reduce the salt content in dried fish, such that the salt content in dried fish keeps a 1-5% range, beneficial to the smooth implementation of mixed fermentation.

Further, the mixed starter culture in the step (3) has an inoculum size of 10-20 mL/100 g dried fish; and the inoculation way is spray or rolling and rubbing.

Further, the fermentation conditions in the step (3) are as follows: sealed fermentation is performed for 24 h at a fermentation temperature of 20° C.

Further, the preparation method of the dried fish in the step (1) is as follows: taking and scaling off freshwater fish, then dissecting and killing, and pickling the fish with salt which is 15%-20% weight of the fish body for 12 h after being cleaned; then cleaning the pickled fish with running water, draining off and drying the fish to control a water content to 35%.

Further, the drying temperature is 40° C.

After being rinsed with fresh water, the salt content (7-10%) in the dried fish prepared herein is 1-5%. The present invention can further provide better fermentation conditions for the mixed starter culture while preserving fresh to remove the fishy smell and improve aroma, achieving a more delicate flavor, thereby further improving the fresh aroma and taste of the dried fish prepared by mixed fermentation.

In a further aspect, the present invention provides a preparation method of the above mixed starter culture, mainly including the following steps:

(I) activating Bifidobacterium species, Lactobacillus acidophilus, Lactobacillus casei, Streptococcus thermophilus and Bifidobacterium lactis, respectively;

(II) respectively performing domestication culture on the activated bacterial solutions of Bifidobacterium species, Lactobacillus acidophilus, Lactobacillus casei, Streptococcus thermophilus and Bifidobacterium lactis to separately obtain bacterial solutions of Bifidobacterium species, Lactobacillus acidophilus, Lactobacillus casei, Streptococcus thermophilus and Bifidobacterium lactis having a concentration of 10⁸-10⁹ cfu/mL; and

(III) mixing the bacterial solutions of Bifidobacterium species, Lactobacillus acidophilus, Lactobacillus casei, Streptococcus thermophilus and Bifidobacterium lactis according to a volume ratio of 1:1:1:2 to prepare into a mixed starter culture.

Further, the activating culture medium in the step (I) is an MRS culture medium; and the domestication culture medium in the step (II) consists of 50% MRS culture medium and 50% fish broth.

In a further aspect, the present invention provides a use of a mixed starter culture in the preparation of a formulation for increasing a content of a flavor substance and/or a delicious substance of dried fish. The mixed starter culture includes Bifidobacterium species, Lactobacillus acidophilus, Lactobacillus casei, Streptococcus thermophilus and Bifidobacterium lactis; the flavor substance includes one or more of 5-hexyldihydro-2(3H)-furanone, 3-heptyldihydro-5-methyl-2(3H)-furanone, nonanal, and hexadecanal; the delicious substance includes glutamic acid and/or aspartic acid.

Further, in the mixed starter culture, Bifidobacterium species, Lactobacillus acidophilus, Lactobacillus casei, Streptococcus thermophilus and Bifidobacterium lactis have a volume ratio of 1:1:1:2; and the mixed starter culture has a concentration of bacterium of 10⁸-10⁹ cfu/mL.

The present invention has the following beneficial effects:

(1) the dried fish prepared by the mixed fermentation of Bifidobacterium species, Lactobacillus acidophilus, Lactobacillus casei, Streptococcus thermophilus and Bifidobacterium lactis can obviously improve the delicious taste and aroma, such that the fish tastes not hard any more, soft and delicious;

(2) the content of Bifidobacterium lactis in the mixed starter culture is increased to further improve the fermentation synergy of the five bacteria to obviously increase the content of flavor substances, namely, 5-hexyldihydro-2(3H)-furanone, 3-heptyldihydro-5-methyl-2(3H)-furanone, nonanal, and hexadecanal as well as the delicious substances, namely, glutamic acid and aspartic acid, thus achieving a better taste;

(3) salt content in the dried fish is 1-5% after being rinsed with fresh water; therefore, the present invention can further provide better fermentation conditions for the mixed starter culture while preserving fresh to remove the fishy smell and improve aroma, achieving a more delicate flavor, thereby further improving the fresh aroma and taste of the dried fish prepared by mixed fermentation;

(4) the inoculum size and fermentation conditions of mixed fermentation are optimized to further promote the fermentation effect.

### BRIEF DESCRIPTION OF THE DRAWINGS

FIG. 1 is a gas chromatography-mass spectrometry diagram of a dried fish prepared by mixed fermentation in Example 6;

FIG. 2 is a gas chromatography-mass spectrometry diagram of non-fermented dried fish in Example 6.

### DETAILED DESCRIPTION OF THE EMBODIMENTS

The present invention will be further described by reference to the examples. It should be indicated that the following examples are aimed at understanding the present invention, but not construed as limiting the present invention. Reagents in this example are known products purchased from the market available.

### Example 1 Preparation of the Mixed Fermented Dried Fish

The preparation method of the mixed fermented dried fish provided in this example is as follows:

(1) Preparation of Dried Fish:

a freshwater fish (grass carp) was taken, scaled off, dissected and killed, pickled with salt which was 20% weight of the fish body for 12 h after being cleaned; then the pickled fish was cleaned with running water to remove residues and surface stains, and then drained off and dried at 40° C. to control a water content to 35% around; the dried fish was cut into pieces having a side length of about 5 cm, and salt content was about 10%.

(2) Rinsing the Dried Fish with Fresh Water:

the dried fish was taken and rinsed with fresh water for 1 h, and water was exchanged twice, where, the ratio of raw material to water was 1:15. the rinsed dried fish was drained off to remove surface water, and then put to a constant temperature drying oven at 40° C. for drying until 1.1-1.2 times of the raw dried fish before rinsing with fresh water; salt content in the dried fish after being rinsed and dried was about 2%;

(3) Inoculation with the Mixed Starter Culture for Fermentation:

a. Preparation of the Mixed Starter Culture:

Bifidobacterium species powder (purchased from China Center of Industrial Culture Collection (CICC) No. 21711), Lactobacillus acidophilus powder (purchased from CICC No. 20244), Lactobacillus casei (purchased from CICC No. 20241), Streptococcus thermophilus (purchased from Mufan Biotechnology Company No. MF-001031) and Bifidobacterium lactis powder (purchased from Mufan Biotechnology Company No. MF-001040) were respectively taken and inoculated onto MRS culture media (purchased from Qingdao Hopebio, type HB0384-1) with an inoculum size of 5%, and then cultured over the night at 30° C.;

the activated bacterial solutions of Bifidobacterium species, Lactobacillus acidophilus, Lactobacillus casei, Streptococcus thermophilus and Bifidobacterium lactis were then respectively taken and inoculated onto domestication culture media (50% MRS culture media and 50% fish broth) for domestication culture for 24 h at 30° C. with an inoculum size of 10% to respectively obtain the bacterial solutions of Bifidobacterium species, Lactobacillus acidophilus, Lactobacillus casei, Streptococcus thermophilus and Bifidobacterium lactis having a concentration of 10⁸-10⁹ cfu/m L;

the bacterial solutions of Bifidobacterium species, Lactobacillus acidophilus, Lactobacillus casei, Streptococcus thermophilus and Bifidobacterium lactis were mixed according to a volume ratio of 1:1:1:2 to prepare into a mixed starter culture;

b. Mixed Fermentation

the mixed starter culture was inoculated to the rinsed dried fish with an inoculum size of 15 mL/100 g dried fish via spray or rolling and rubbing, and stirred to be mixed well; then the mixture was seal fermented for 24 h at 20° C. the fermented dried fish was put to a constant temperature drying oven for drying at 40° C. until the original weight of the dried fish before rinsing with fresh water; then, the fermented dried fish product was obtained.

### Example 2 Influences of Different Fermented Starters on the Prepared Dried Fish

In this example, the dried fish was prepared by the method provided in Example 1; during fermentation, different starters (the starters used are shown in Table 1; starters were mixed according to the same volume in the combination of bacterial solution) were inoculated to the dried fish with an inoculum size of 15 mL/100 g dried fish and concentration of 10⁸-10⁹ cfu/mL. The contents of flavor substances, 5-hexyldihydro-2(3H)-furanone (peach flavor), 3-heptyldihydro-5-methyl-2(3H)-furanone (green onion/garlic flavor), nonanal (waxy and fat flavor), hexadecanal (flower and wax flavor), and delicious substances, glutamic acid and aspartic acid in the prepared dried fish as well as the fishy smell and taste were detected. The content of flavor substances was detected by gas chromatography-mass spectrography, and the content of delicious substances was reflected by the content of free amino acids; fishy smell and taste were subjected to comprehensive evaluation after being tasted by 30 volunteers, thereby exploring the influences of different fermented starters on the flavor substances, delicious substances and flavor of the prepared dried fish. The results are shown in Table 1.

TABLE 1
Influences of different fermented starters on the prepared dried fish

| | | 3-heptyldihydro- | | | | | | |
|---|---|---|---|---|---|---|---|---|
| | 5-hexyldihydro- | 5-methyl-2(3H)- | Nonanal | Hexadecanal | Glutamic | Aspartic | Fishy | |
| Starter | 2(3H)-furanone | furanone | (%) | (%) | acid (%) | acid (%) | smell | Taste |
| Bifidobacterium | 0.71 | 1.58 | 0.65 | 1.77 | 8.5 | 6.7 | Slightly | Soft |
| species | | | | | | | fishy | |
| Lactobacillus | 0.83 | 1.82 | 0.72 | 1.83 | 9.1 | 7.5 | Slightly | Soft |
| acidophilus | | | | | | | fishy | |
| Lactobacillus | 0.94 | 1.92 | 0.83 | 2.32 | 8.7 | 7.2 | No fishy | Slightly |
| casei | | | | | | | smell | hard |
| Streptococcus | 1.01 | 1.75 | 0.66 | 1.64 | 8.5 | 8.9 | Slightly | Slightly |
| thermophilus | | | | | | | fishy | hard |
| Bifidobacterium | 1.52 | 2.46 | 0.88 | 3.08 | 9.8 | 9.5 | No fishy | Soft |
| lactis | | | | | | | smell | |
| Bifidobacterium | 1.54 | 2.51 | 0.87 | 3.09 | 9.7 | 9.8 | No fishy | Soft |
| lactis + | | | | | | | smell | |
| Bifidobacterium | | | | | | | | |
| species | | | | | | | | |
| Bifidobacterium | 1.53 | 2.55 | 0.85 | 3.11 | 9.5 | 10.7 | No fishy | Soft |
| lactis + | | | | | | | smell | |
| Lactobacillus | | | | | | | | |
| acidophilus | | | | | | | | |
| Bifidobacterium | 1.84 | 2.65 | 0.91 | 3.17 | 10.4 | 12.3 | No fishy | Slightly |
| lactis + | | | | | | | smell | soft |
| Lactobacillus | | | | | | | | |
| casei | | | | | | | | |
| Bifidobacterium | 1.65 | 2.63 | 0.89 | 3.15 | 9.8 | 11.3 | No fishy | Slightly |
| lactis + | | | | | | | smell | soft |
| Streptococcus | | | | | | | | |
| thermophilus | | | | | | | | |
| Bifidobacterium | 1.93 | 2.96 | 0.87 | 3.30 | 10.9 | 12.5 | No fishy | More soft |
| lactis + | | | | | | | smell | and waxy |
| Bifidobacterium | | | | | | | | |
| species + | | | | | | | | |
| Lactobacillus | | | | | | | | |
| acidophilus | | | | | | | | |
| Bifidobacterium | 2.01 | 3.24 | 0.98 | 3.58 | 11.8 | 14.4 | No fishy | Soft |
| lactis + | | | | | | | smell | |
| Lactobacillus | | | | | | | | |
| casei + | | | | | | | | |
| Streptococcus | | | | | | | | |
| thermophilus | | | | | | | | |
| Bifidobacterium | 1.94 | 3.05 | 0.89 | 3.21 | 11.1 | 12.8 | No fishy | More soft |
| lactis + | | | | | | | smell | and waxy |
| Bifidobacterium | | | | | | | | |
| species + | | | | | | | | |
| Lactobacillus | | | | | | | | |
| acidophilus + | | | | | | | | |
| Lactobacillus | | | | | | | | |
| casei | | | | | | | | |
| Bifidobacterium | 2.25 | 3.87 | 1.01 | 4.03 | 12.6 | 16.8 | No fishy | Very |
| lactis + | | | | | | | smell | delicious |
| Bifidobacterium | | | | | | | | and waxy |
| species + | | | | | | | | |
| Lactobacillus | | | | | | | | |
| acidophilus + | | | | | | | | |
| Lactobacillus | | | | | | | | |
| casei + | | | | | | | | |
| Streptococcus | | | | | | | | |
| thermophilus | | | | | | | | |

It can be seen from Table 1 that the fermentation with a single bacterium may slightly increase the flavor substances and delicious substances in dried fish, but the effect is not obvious; the result is that the dried fish tastes not enough delicious and waxy, and sometimes slightly hard, and even the fishy smell of the dried fish may not be completely removed; the effect of Bifidobacterium lactis in improving flavor substances and delicious substances in dried fish is slightly better than other bacteria, and the effect of removing fishy smell is better slightly.

It can be seen that during the combined fermentation of several bacteria, the combination of the five bacteria according to the same volume for mixed fermentation has an excellent effect of improving the quality of dried fish, obviously better than the mixed fermentation effects of the two, three and four bacteria. The reason may be because the five bacteria create a synergistic effect during mixed fermentation to promote the production of more flavor and delicious substances in the pickled fish. The contents of 5-hexyldihydro-2(3H)-furanone, 3-heptyldihydro-5-methyl-2(3H)-furanone, nonanal, hexadecanal, glutamic acid and aspartic acid are up to 2.25%, 3.87%, 1.01%, 4.03%, 12.6% and 16.8%. The dried fish tastes very good, delicious and waxy.

### Example 3 Influences of the Content of Bifidobacterium lactis in the Mixed Starter Culture on the Prepared Dried Fish

In this example, the dried fish was prepared by the method provided in Example 1; during mixed fermentation, the proportional relation of Bifidobacterium lactis in the mixed bacterial solution was changed (see details in Table 2); then, the mixed bacterial solution was respectively inoculated to dried fish for mixed fermentation with an inoculum size of 15 mL/100 g dried fish and concentration of 10⁸-10⁹ cfu/mL. The contents of flavor substances, 5-hexyldihydro-2(3H)-furanone (peach flavor), 3-heptyldihydro-5-methyl-2(3H)-furanone (green onion/garlic flavor), nonanal (wax and fat flavor), hexadecanal (flower and wax flavor), and delicious substances, glutamic acid and aspartic acid in the prepared dried fish as well as the fishy smell and taste were detected. The detection method is the same as that in Example 2. The results are shown in Table 2.

TABLE 2
Influences of the proportional relation of Bifidobacterium lactis in the mixed starter culture on the prepared dried fish

| Bifidobacterium | | | | | | | | |
|---|---|---|---|---|---|---|---|---|
| species:Lactobacillus | | | | | | | | |
| acidophilus:Lactobacillus | | | | | | | | |
| casei:Streptococcus | | 3-heptyldihydro- | | | | | | |
| thermophilus:Bifidobacterium | 5-hexyldihydro- | 5-methyl-2(3H)- | Nonanal | Hexadecanal | Glutamic | Aspartic | Fishy | |
| lactis | 2(3H)-furanone | furanone | (%) | (%) | acid (%) | acid (%) | smell | Taste |
| 1:1:1:1:1 | 2.25 | 3.87 | 1.01 | 4.03 | 12.6 | 16.8 | No fishy | Very |
| | | | | | | | smell | delicious |
| | | | | | | | | and waxy |
| 1:1:1:1:1.5 | 2.28 | 3.98 | 1.01 | 4.14 | 13.1 | 17.0 | No fishy | Very |
| | | | | | | | smell | delicious |
| | | | | | | | | and waxy |
| 1:1:1:1:2 | 2.34 | 4.03 | 1.02 | 4.27 | 13.5 | 17.7 | No fishy | Very |
| | | | | | | | smell | delicious, |
| | | | | | | | | waxy and |
| | | | | | | | | delicate |
| 1:1:1:1:3 | 2.01 | 2.94 | 0.95 | 3.24 | 10.7 | 15.5 | No fishy | Too soft |
| | | | | | | | smell | |

It can be seen from Table 2 that the proportional relation of Bifidobacterium lactis in the mixed bacterial solution has greater influences on the quality of dried fish; when the ratio of Bifidobacterium species: Lactobacillus acidophilus: Lactobacillus casei: Streptococcus thermophilus: Bifidobacterium lactis is up to 1:1:1:1:2, the content of flavor and delicious substances in the pickled fish is up to the maximum; and the contents of 5-hexyldihydro-2(3H)-furanone, 3-heptyldihydro-5-methyl-2(3H)-furanone, nonanal, hexadecanal, glutamic acid and aspartic acid are respectively up to 2.34%, 4.03%, 1.02%, 4.27%, 13.5% and 17.7%. The dried fish has an excellent taste, free of fishy smell, very delicious, waxy and delicate. When the ratio of Bifidobacterium lactis is further increased, the content of various flavor and delicious substances significantly decreases; moreover, the prepared dried fish tastes too soft and not very good. Therefore, the ratio of Bifidobacterium species, Lactobacillus acidophilus, Lactobacillus casei, Streptococcus thermophilus and Bifidobacterium lactis is controlled to 1:1:1:1:2, thus preparing dried fish with better quality.

### Example 4 Influences of Salt Content on the Prepared Dried Fish after being Rinsed with Fresh Water

In this example, the dried fish was prepared by the method provided in Example 1; the salt content in the dried fish was controlled by controlling the rinsing time. It is proved by experiments that the dried fish has a salt content of 6% after being rinsed with fresh water for 30 min, has a salt content of 2% after being rinsed with fresh water for 60 min, and has a salt content of 0.1% after being rinsed with fresh water for 90 min; and then the dried fish was subjected to mixed fermentation respectively. The contents of flavor substances, 5-hexyldihydro-2(3H)-furanone (peach flavor), 3-heptyldihydro-5-methyl-2(3H)-furanone (green onion/garlic flavor), nonanal (waxy and fat flavor), hexadecanal (flower and wax flavor), and delicious substances, glutamic acid and aspartic acid in the prepared dried fish as well as the fishy smell and taste were detected. The detection method is the same as that in Example 2. The results are shown in Table 3.

TABLE 3
Influences of salt content on the prepared dried fish after being rinsed with fresh water

| | | 3-heptyldihydro- | | | | | | |
|---|---|---|---|---|---|---|---|---|
| Rinsing | 5-hexyldihydro- | 5-methyl-2(3H)- | Nonanal | Hexadecanal | Glutamic | Aspartic | Fishy | |
| time | 2(3H)-furanone | furanone | (%) | (%) | acid (%) | acid (%) | smell | Taste |
| 30 min | 0.79 | 1.34 | 0.85 | 1.97 | 8.1 | 10.7 | No fishy | Slightly |
| | | | | | | | smell | hard |
| 60 min | 2.34 | 4.03 | 1.02 | 4.27 | 13.5 | 17.7 | No fishy | Very |
| | | | | | | | smell | delicious, |
| | | | | | | | | waxy and |
| | | | | | | | | delicate |
| 90 min | 2.17 | 3.02 | 0.98 | 3.75 | 12.4 | 15.6 | No fishy | Too soft |
| | | | | | | | smell | |

It can be seen from Table 3 that the rinsing time has greater influences on the quality of the dried fish. The reason is that the salt content in the dried fish varies from the rinsing time; and the salt content in the dried fish will directly influence the effect of the mixed fermentation. When the rinsing time is short (30 min), the salt content in the dried fish is higher, and if the mixed fermentation is performed at this time, the mixed fermentation may be inhibited, leading to not enough flavor and delicious substances in the dried fish. When the rinsing time is too long (90 min), the salt content in the dried fish is very low, and if the mixed fermentation is performed at this time, the quality of the prepared dried fish declines. It can be seen that the rinsing time is controlled to keep a certain salt content in the dried fish, which is also an important condition to prepare mixed fermented dried fish with high quality; that is, the rinsing time may provide more suitable fermentation conditions for the mixed fermentation, thus promoting the increase of flavor and delicious substances in the dried fish, and improving taste. Therefore, the rinsing time needs to be controlled to 60 min.

### Example 5 Influences of Inoculum Size of the Mixed Starter Culture on the Prepared Dried Fish

In this example, the dried fish was prepared by the method provided in Example 1; the inoculum size of the mixed starter culture was respectively selected from 5, 10, 15, 20 and 25 mL/100 g (as shown in Table 4. The contents of flavor substances, 5-hexyldihydro-2(3H)-furanone (peach flavor), 3-heptyldihydro-5-methyl-2(3H)-furanone (green onion/garlic flavor), nonanal (wax and fat flavor), hexadecanal (flower and wax flavor), and delicious substances, glutamic acid and aspartic acid in the prepared dried fish as well as the fishy smell and taste were detected. The detection method is the same as that in Example 2. The results are shown in Table 4.

TABLE 4
Influences of inoculum size of the mixed starter culture on the prepared dried fish

| Inoculum | | | | | | | | |
|---|---|---|---|---|---|---|---|---|
| size | | 3-heptyldihydro- | | | | | | |
| (mL/100 g | 5-hexyldihydro- | 5-methyl-2(3H)- | Nonanal | Hexadecanal | Glutamic | Aspartic | Fishy | |
| dried fish) | 2(3H)-furanone | furanone | (%) | (%) | acid (%) | acid (%) | smell | Taste |
| 5 | 1.35 | 1.89 | 0.89 | 2.57 | 10.7 | 13.6 | Slightly | Slightly |
| | | | | | | | fishy | hard |
| 10 | 2.24 | 3.97 | 0.99 | 4.01 | 12.9 | 16.1 | No fishy | Very |
| | | | | | | | smell | delicious, |
| | | | | | | | | waxy and |
| | | | | | | | | delicate |
| 15 | 2.35 | 4.04 | 1.02 | 4.28 | 13.5 | 17.6 | No fishy | Very |
| | | | | | | | smell | delicious, |
| | | | | | | | | waxy and |
| | | | | | | | | delicate |
| 20 | 2.19 | 3.95 | 0.95 | 4.03 | 12.7 | 16.5 | No fishy | Very |
| | | | | | | | smell | delicious, |
| | | | | | | | | waxy and |
| | | | | | | | | delicate |
| 25 | 2.01 | 3.58 | 0.89 | 3.95 | 12.0 | 15.7 | No fishy | Too soft |
| | | | | | | | smell | |

It can be seen from Table 4 that a proper inoculum size is selected to obtain better quality of dried fish. When the inoculum size of the mixed starter culture is 10-20 mL/100 g dried fish, the prepared dried fish has a higher content of flavor and delicious substances, tastes delicious, waxy and delicate, free of fishy smell. The optimal inoculum size is 15 mL/100 g dried fish; and at this time, the prepared dried fish has the highest content of flavor and delicious substances, and tastes excellent.

### Example 6 Comparison Between the Dried Fish Prepared in this Present Invention and the Dried Fish without Mixed Fermentation

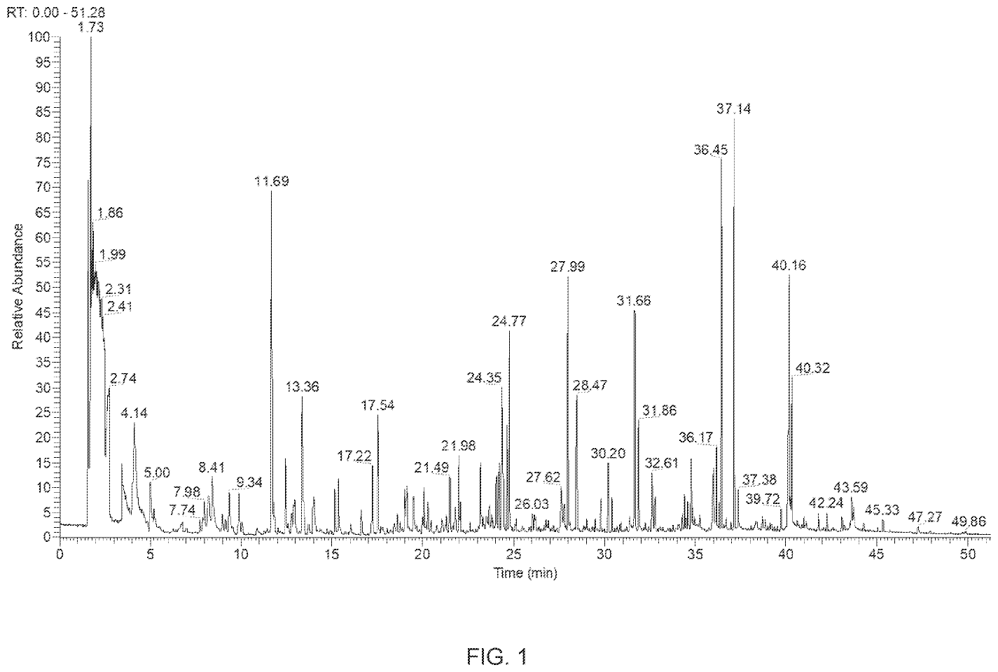

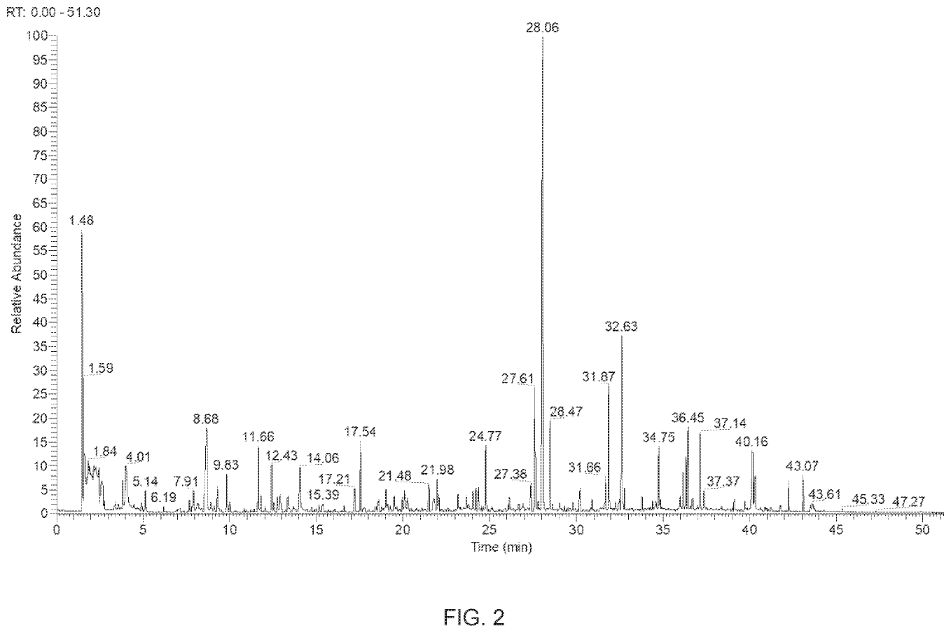

In this example, the dried fish was prepared by the method provided in Example 1; the dried fish without mixed fermentation (the dried fish prepared in the step one of Example 1) served as a control group for comparison. The content of volatile substances in the two kinds of dried fish was detected by gas chromatography-mass spectrometry; and the content of delicious substances was detected (reflected by the content of free amino acids); color, smell, taste and state of the two groups of dried fish were scored. There are 10 scores in total, more than 8 represents excellent quality (white and light yellow, aromatic and delicate flavor, good taste, slightly sweet residual taste, soft and waxy and moderate meat quality); more than 6 represents good quality, and less than 6 represents poor quality (brown, obvious fishy smell, spicy flavor, bitter taste of residual taste, too hard or too soft meat). The gas chromatography-mass spectrometry results are shown in FIGS. 1-2 (FIG. 1 is a detection diagram of the dried fish prepared by mixed fermentation provided in Example 1; FIG. 2 is a detection diagram of the dried fish in the control group) and Tables 5-6 (Table 5 shows the detection results of the dried fish prepared by mixed fermentation provided in Example 1; Table 6 shows the detection results of the dried fish in the control group). Detection results of the delicious substances, and score results of the color, smell, taste and state are shown in Table 7.

TABLE 5
Chemical contents of volatile substances in
the dried fish prepared by mixed fermentation

| | | | | Relative | |
|---|---|---|---|---|---|
| | | | Aroma | percentage | |
| | | Name of volatile substances | characteristics | (%) | |
| Ketone | 5 | 5-hexyldihydro-2(3H)-furanone | Peach flavor | 2.34 | 7.67 |
| | | 3-heptyldihydro-5-methyl-2(3H)-furanone | Green onion/ | 4.03 | |
| | | | garlic flavor | | |
| | | 2-nonanone | Fruity and sweet | 0.39 | |
| | | 2-hendecanone | Wax and fat | 0.59 | |
| | | | flavor | | |
| | | 2-Tridecanone | Milky and | 0.32 | |
| | | | coconut flavor | | |
| Benzene | 1 | 3-allyl-6-metoxybenzene | | 2.7 | 2.7 |
| Acid | 2 | n-palmitic acid | | 3.65 | 4.5 |
| | | Myristic acid | | 0.85 | |
| Ester | 2 | Ethyl cetylate | Weak waxy and | 1.32 | 2.11 |
| | | | cream flavor | | |
| | | N-butyl-isooctyl phthalate | Orange flavor | 0.79 | |
| Alkene | 1 | Styrene | Slightly sweet | 0.69 | 0.69 |
| Alcohol | 5 | 1-octene-3-ol | Mushroom flavor | 4.46 | 7.43 |
| | | 2-phenethyl alcohol | Rose flavor | 0.62 | |
| | | 3-octanol | | 1.01 | |
| | | 1-octanol | Strong grease | 0.59 | |
| | | | smell and rose | | |
| | | | flavor | | |
| | | 2-octene-1-ol | | 0.75 | |
| Aldehyde | 3 | Nonanal | Waxy and fat | 1.01 | 6.37 |
| | | | flavor | | |
| | | Hexadecanal | Weak flowery | 4.27 | |
| | | | and wax flavor | | |
| | | N-undecylic aldehyde | Fat and flowery | 1.09 | |
| | | | flavor | | |
| Others | 1 | Dimethyl trisulfide | | 2.27 | 2.27 |
| Alkane | 9 | Dodecane | | 1.69 | 11.96 |
| | | N-decane | | 0.69 | |
| | | Nonadecane | | 0.79 | |
| | | Octamethylcyclotetrasiloxane | | 0.75 | |
| | | N-hexadecane | | 0.72 | |
| | | Dodecamethylcyclohexasiloxane | | 0.72 | |
| | | Cetylcyclooctasiloxane | | 1.59 | |
| | | Hexamethylcyclotrisiloxane | | 3.19 | |
| | | Tetradecamethylcycloheptasiloxane | | 1.82 | |

TABLE 6
Chemical contents of volatile substances of the dried fish in the control group

| | | | | Relative | |
|---|---|---|---|---|---|
| | | | Aroma | percentage | |
| | | Name of volatile substances | characteristics | (%) | |
| Ketone | 3 | 3-octanone | Fruit aroma | 1.15 | 3.6 |
| | | 5-hexyldihydro-2(3H)-furanone | Peach flavor | 0.71 | |
| | | 3-heptyldihydro-5-methyl-2(3H)-furanone | Green onion/ | 1.74 | |
| | | | garlic flavor | | |
| Phenols | 2 | Eugenol | Clove flavor | 14.26 | 14.85 |
| | | Phenol | Special odor, and | 0.59 | |
| | | | highly-diluted | | |
| | | | solution is sweet | | |
| Benzene | 2 | P-xylene | Aromatic flavor | 0.67 | 1.01 |
| | | Ethylbenzene | | 0.34 | |
| Acid | 3 | n-palmitic acid | | 1.86 | 2.44 |
| | | Myristic acid | | 0.44 | |
| | | cis-octadecenoic acid | | 0.14 | |
| Ester | 4 | Dibutyl phthalate | Aromatic flavor | 0.6 | 2.49 |
| | | Ethyl myristate | | 1.09 | |
| | | Ethyl cetylate | Orange flavor | 0.58 | |
| | | Methyl palmitate | | 0.22 | |
| Alkene | 2 | Styrene | | 0.72 | 1.37 |
| | | Caryophyllene | Spicy, wood and | 0.65 | |
| | | | orange flavor | | |
| Alcohol | 4 | 1-octene-3-ol | Mushroom | 1.61 | 2.71 |
| | | | flavor | | |
| | | 2-phenethyl alcohol | Rose flavor | 0.52 | |
| | | Hendecanol | | 0.42 | |
| | | 3-octanol | | 0.16 | |
| Aldehyde | 3 | Nonanal | Waxy and fat | 0.63 | 2.77 |
| | | | flavor | | |
| | | Hexadecanal | Weak flowery | 1.66 | |
| | | | and wax flavor | | |
| | | Cis-9-hexadecenal | | 0.48 | |
| Others | 2 | Methoxybenzene oxime | | 3.5 | 6.98 |
| | | Benzpyrole | Highly-diluted | 3.48 | |
| | | | solution is | | |
| | | | fragrant | | |
| Alkane | 14 | Dodecane | | 2.14 | 19.63 |
| | | N-decane | | 1.11 | |
| | | Heptadecane | | 3.94 | |
| | | Octamethylcyclotetrasiloxane | | 0.37 | |
| | | N-tetradecane | | 1.46 | |
| | | N-hexadecane | | 0.49 | |
| | | Dodecamethylcyclohexasiloxane | | 0.29 | |
| | | Dodecamethylcyclohexasiloxane | | 1.15 | |
| | | Cetylcyclooctasiloxane | | 2.07 | |
| | | 8-propoxy cedulane | | 0.35 | |
| | | Hexamethylcyclotrisiloxane | | 1.48 | |
| | | Octadecmethylcyclononasiloxane | | 2.88 | |
| | | Dimethyldiacetoxysilane | | 0.48 | |
| | | Hexadecmethyloctasiloxane | | 1.42 | |

TABLE 7
Detection results of delicious substances
in the two groups of dried fish

| | Glutamic | Aspartic | | | | |
|---|---|---|---|---|---|---|
| Group | acid (%) | acid (%) | Color | Smell | Taste | State |
| Mixed fermented | 13.5 | 17.6 | 9 | 9 | 9 | 9 |
| dried fish | | | | | | |
| Control group | 8.3 | 6.5 | 6 | 5 | 5 | 6 |

It can be seen from FIGS. 1-2 and Tables 5-6 that the dried fish prepared by mixed fermentation provided herein produces a large number of volatile flavor substances to achieve an aromatic flavor and excellent taste based on the original dried fish (control group). It can be seen from Table 7 that the dried fish prepared by mixed fermentation provided herein has obviously improved delicious substances, yellow and bright color, aromatic and fresh flavor, slightly sweet residual taste, soft and waxy meat; and the quality is promoted greatly. The dried fish will be well received by consumers.

The present invention is disclosed above, but the present invention is not limited thereto. Any person skilled in the art may make various alterations and modifications within the spirit and scope of the present invention. Therefore, the protection scope of the present invention shall be subjected to the scope defined in the claims appended.

1. A mixed starter culture of a dried fish, comprising Bifidobacterium species, Lactobacillus acidophilus, Lactobacillus casei, Streptococcus thermophilus and Bifidobacterium lactis. wherein in the mixed starter culture, Bifidobacterium species, Lactobacillus acidophilus, Lactobacillus casei, Streptococcus thermophilus and Bifidobacterium lactis have a volume ratio of 1:1:1:2; and the mixed starter culture has a concentration of bacterium of 10⁸-10⁹ cfu/mL.

2. (canceled)

3. A method for preparing a dried fish, comprising the following steps:
preparing a dried fish: taking and scaling off freshwater fish, then dissecting and killing, and pickling the fish with salt which is 15%-20% weight of the fish body for 12 h after being cleaned; then cleaning the pickled fish with running water, draining off and drying the fish to control a water content to 35%;
rinsing the dried fish with fresh water: subjecting the dried fish to desalting treatment for 1-2 h, and then baking the dried fish at a condition of 40° C., and after baking, the weight of the dried fish being 1.1-1.2 times of the original weight; and
inoculating with the mixed starter culture as claimed in claim 1 for fermentation, wherein the mixed starter culture has an inoculum size of 10-20 mL/100 g dried fish, the inoculation way is spray or rolling and rubbing, and sealed fermentation is performed for 24 h at a fermentation temperature of 20° C.
